(19) United States
(12) Patent Application Publication
(10) Pub. No.: US 2016/0008177 A1
(43) Pub. Date: Jan. 14, 2016

(54) DEVICE AND WRAPPING METHOD FOR ENCLOSING OF PORTIONS OF EXCREMENT, BANDAGING MATERIALS, FEMININE HYGIENE ARTICLES OR THE LIKE IN WRAPPINGS

(71) Applicant: ROTHO BABYDESIGN GMBH, Gorwihl (DE)

(72) Inventor: Fritz Schmitt, Wasserbillig (LU)

(73) Assignee: ROTHO BABYDESIGN GMBH, Gorwihl (DE)

(21) Appl. No.: 14/798,445

(22) Filed: Jul. 13, 2015

(30) Foreign Application Priority Data
Jul. 11, 2014 (DE) 10 2014 010 364.5

(51) Int. Cl. A61F 13/00; A61F 13/551; B65B 31/02; B65B 9/04; B65B 25/00

(52) U.S. Cl. CPC A61F 13/00072; B65B 9/04; B65B 25/00; B65B 31/024; A61F 13/5515; A61F 13/5518

(57) ABSTRACT

Disclosed is a device and a wrapping method for enclosing of portions of excrement, bandaging materials, feminine hygiene articles or the like in wrappings comprising a container or a lid for a container with at least one fill opening to receive at least one object being sealed, and a movement coordinator at least partly received in the container. According to an embodiment, at least two wrapping materials are provided, which are arranged before or before and after the fill opening.

### CROSS REFERENCE TO RELATED APPLICATIONS

The present application claims priority to German Patent Application No. 10 2014 010 364.5, filed Jul. 11, 2014, entitled DEVICE AND PACKAGING METHOD FOR PORTION-WISE ENCLOSING OF EXCREMENT, BANDAGING MATERIALS, FEMALE HYGIENE ARTICLES OR THE LIKE IN PACKAGES, the entirety of which is hereby incorporated by reference.

### COPYRIGHT NOTICE

A portion of the disclosure of this patent document contains material that is subject to copyright protection. The copyright owner has no objection to the facsimile reproduction by anyone of the patent document or the patent disclosure as it appears in the Patent and Trademark Office patent files or records, but otherwise reserves all copyrights whatsoever.

### SUMMARY

The present disclosure pertains to a device and a wrapping method for enclosing of portions of excrement, bandaging materials, feminine hygiene articles or the like in wrappings.

From EP 1 324 919 B1 there is known a device for the separate sealing of a plurality of objects, enabling a receiving of contaminated material for wrapping through a rotatable ring magazine.

The present device provides an improved solution for the wrapping of such objects as excrement, bandaging materials, feminine hygiene articles, diapers or the like. In particular, a need for improvement is seen in regard to the consumption of material, the tight closure technique by portions, and the handling.

Described are embodiments for the enclosing of portions of excrement, bandaging materials, feminine hygiene articles or the like in wrappings and have: a container and/or a lid for a container with at least one fill opening to receive at least one object being sealed, namely, a wrapping item, and moreover a movement coordinator at least partly received in the container and/or the lid for a propelling of the object in at least one direction of movement.

According to embodiments, the device has at least one magazine for dispensing of at least two wrapping materials. At least one surface region of the movement coordinator is arranged in the direction of movement toward the magazine, as is the fill opening. The movement coordinator passes at least with its surface region through the region behind the magazine in which the fill opening is arranged during the movement.

A wrapping method is disclosed, according to embodiments of the invention, for enclosing portions of excrement, bandaging materials, feminine hygiene articles or the like in wrappings with a transport device, which is preferably part of the movement coordinator, for a first wrapping material. The transport device forms together with the first wrapping material a receiving recess. The wrapping method furthermore works with a second wrapping material, which is preferably stockpiled in a sealing mechanism, which is intended to close the receiving recess, and a body which can move for the sealing. A single movement of the body ensures first of all a removal of the first wrapping material from the first magazine, secondly and preferably a sealing of the material being wrapped that has been inserted into the receiving recess, and thirdly but always a sealing of the wrapping by means of the second wrapping material from the same magazine or a second magazine. This is done in that the sealing mechanism, preferably cooperating with the movement coordinator, brings both wrapping materials in contact around the receiving recess in a sealing zone.

The use of two magazines allows a thrifty use of low-cost wrapping material which can be separately heated with different properties, so that on the whole the trash volume produced is kept as low as possible. The use of two wrapping materials instead of a single wrapping material moreover makes it possible to influence the wrapping during the wrapping process, especially a sealing of the wrapping. The disclosed method and embodiments enable a comfortable handling with, for example, only a single stroke, while this stroke when the work steps are divided over the periphery of the drum or the body removes the wrapping material and prepares it for reception, while at the same time an inserted material is wrapped as a genuine portion.

According to embodiments, only the size of the object determines the size of the subsequent wrapping. In embodiments, the device accomplishes the aforementioned compressing of the object before the wrapping is closed. After the closing, the wrapping is configured to be capable, through a gas tightness of the wrapping materials themselves and their connection, to fix or almost freeze in the compressing of the object so that the wrapping withstands forces of the object once again striving to expand and optionally the additional forces of gases escaping due to space requirements, which are also not released through the wrapping.

According to embodiments, the axis of rotation of the drum is oriented at least approximately horizontally or vertically.

The horizontal orientation of the drum achieves a mobility of the central process body by means of a vertical stroke, which is performed for example by means of a foot pedal and the pressure of the human leg. This direction of application of force corresponds especially well to the reception possibilities, for example, when the device is set up on a level floor.

According to another embodiment of the invention, the magazines are arranged with wrapping rotation axes parallel to the axis of rotation of the rotatable drum.

If all axis orientations are placed parallel to each other, there is a minimal consumption of wrapping material.

According to another embodiment of the device, a gusset is formed between an abutment fastened or formed in the container and the drum. The design of such a tapering region in the transport path of the wrapping, especially during the closure process, allows the kinetic force of the central process body to accomplish the sealing and improve the sealing of the portion.

According to another embodiment of the device, the fill opening is coordinated with at least one bottom arranged in the drum, which can be locked by being arranged congruent to the surface of the fill opening, depending on the position of the drum.

This improves the handling and safeguards the result of the wrapping. The wrapping material does not have to withstand alone the forces during the filling with the wrapping item; it is supported by the bottom.

In embodiments, the bottom facing the direction of rotation in a radial cross section has a smaller inlet radius and a larger outlet radius.

In this way, the bottom also furthers the sealing during the transport of the wrapping.

According to another embodiment of the device, the drum has a plurality of receiving recesses bordered by sealing zones distributed around its circumference or its running surface, the sealing zones being partial surfaces of an imaginary circumferential surface of the drum.

Such sealing zones, preferably with knobs in the sealing surfaces of the drum, besides enhancing the sealing capacity can also make the transport of the wrapping material at least more precise.

According to another embodiment of the device, a pressure roller of the second magazine is an abutment for the sealing of the wrapping item and a sealing pressure surface at the same time.

This can provide benefits such as material savings and free movement.

According to another embodiment of the device, at least one ejector is arranged in a central region of the drum and the drum is configured to be at least partially radially open at the axial middle. The ejector thus reaches the wrapped material in the peripheral region, coming after the second magazine in the rotation direction, and ejects it. In embodiments, the wrapping deflects the ejector against the force of a spring. This arrangement can improve operating safety.

According to other embodiments of the device, the container has at least one of the following features:

can be sealed gas-tight,

can be closed, in particular, with a combination lock,

containing a counter and an interface for data transmission,

having a display to show the full amount and/or danger of odor,

having a camera,

having a trap bowl for moisture, especially with an odor blocking layer, especially glycerin,

double-walled with absorption material between the walls,

having a lighting fixture and an energy storage, the energy storage being chargeable during the driving of the drum and/or by solar energy,

having an atmosphere on the inside, especially a sterilizing gas,

having a trap door with clamping lip as a seal,

having a window with magnifying glass for intensive illumination of the container volume.

According to a wrapping method, a cold welding occurs around the entire receiving recess in the sealing zone, in order to hermetically block off the sealed wrapping item from the surroundings.

According to another embodiment of the wrapping method of the invention, the sealing occurring after the wrapping process is first intensified by a shrinkage process, wherein said shrinkage is triggered by a contact between wrapping material and wrapping item. According to an embodiment, a gas tightness of the wrapping materials occurs only after the shrinkage process.

According to another embodiment of the wrapping method, a single driving stroke is required for each sealed portion of wrapping items, which acts on the transport device.

According to another embodiment of the wrapping method of the invention, the driving of the body entrains an active evacuating mechanism, especially a pump. A connection between the wrapping and the evacuating mechanism remains open until a rear end of the wrapping is sealed.

It can be advantageous for example to avoid oxidation extensively from the standpoint of storage and/or utilization, depending on the wrapping item.

In embodiments, devices or wrapping methods are outfitted with materials that are totally biodegradable.

These features can be associated with the following benefits: no odor nuisance; simple function, especially one-hand attendance; storage possibilities of the wrapped items for up to 3 weeks; use of environmentally safe materials. For example, diapers wrapped in this way according to a device of the invention can be prepared for composting or trash incineration, while avoiding losses.

Before the following embodiments of the invention are described more closely, it should first of all be noted that the invention is not limited to the described components or the described steps of the method. Neither does the terminology used constitute any limitation, but instead is merely exemplary in nature. Insofar as the singular is used afterwards in the description and the claims, each time the plural is also included, unless the context explicitly precludes this.

Although the invention is described in detail in the drawings and the preceding representation, the representations are illustrative and exemplary and not to be understood as limiting. In particular, the choice of the proportions of the individual elements as shown in the drawings should not be interpreted as being required or limiting in nature. Furthermore, the invention in particular is not confined to the sample embodiments discussed. Other variants of the invention and their execution will emerge to the person skilled in the art from the preceding disclosure, the figures, and the protection claims.

Terms used in the protection claims such as “comprise”, “have”, “contain” and the like do not rule out further elements or steps. The use of the indefinite article does not rule out a plurality. A single mechanism can perform the functions of several of the units or mechanisms mentioned in the claims.

### BRIEF DESCRIPTION OF THE DRAWINGS

Embodiments are illustrated in the figures of the accompanying drawings, which are meant to be exemplary and not limiting, and in which like references are intended to refer to like or corresponding things.

FIG. 1 shows a side view along a radial line of the interior of a device according to the invention in a sample embodiment, namely, a drum with magazine rolls for wrapping material arranged above and below.

FIG. 2a shows a cross section of an embodiment of a radial section through the device according to the invention along section II-II of FIG. 1, after inserting a wrapping item.

FIG. 2b shows the embodiment of FIG. 2a during the sealing of the wrapping item.

FIG. 3a shows a cross section of an embodiment of another radial section through the device according to the invention along section III-III of FIG. 1, after the sealing and shortly before contact with an ejector.

FIG. 3b shows the embodiment of FIG. 3a after an ejecting with the ejector relaxed.

FIG. 4 shows a side view of a schematically represented drum according to the embodiment previously shown in FIGS. 1 to 3 with an equally schematic representation of a drive variant.

FIG. 5 shows a perspective exterior view of an embodiment of the device per the sample embodiment of the previous FIGS. 1 to 4.

FIG. 6 shows a perspective detail view of the parts of the embodiment represented in FIG. 1.

### DETAILED DESCRIPTION OF THE EMBODIMENTS

Various embodiments now will be described more fully hereinafter with reference to the accompanying drawings, which form a part hereof, and which show, by way of illustration, specific embodiments by which the invention may be practiced. The embodiments may, however, be embodied in many different forms and should not be construed as limited to the embodiments set forth herein; rather, these embodiments are provided so that this disclosure will be thorough and complete, and will fully convey the scope of the embodiments to those skilled in the art. The following detailed description is, therefore, not to be taken in a limiting sense.

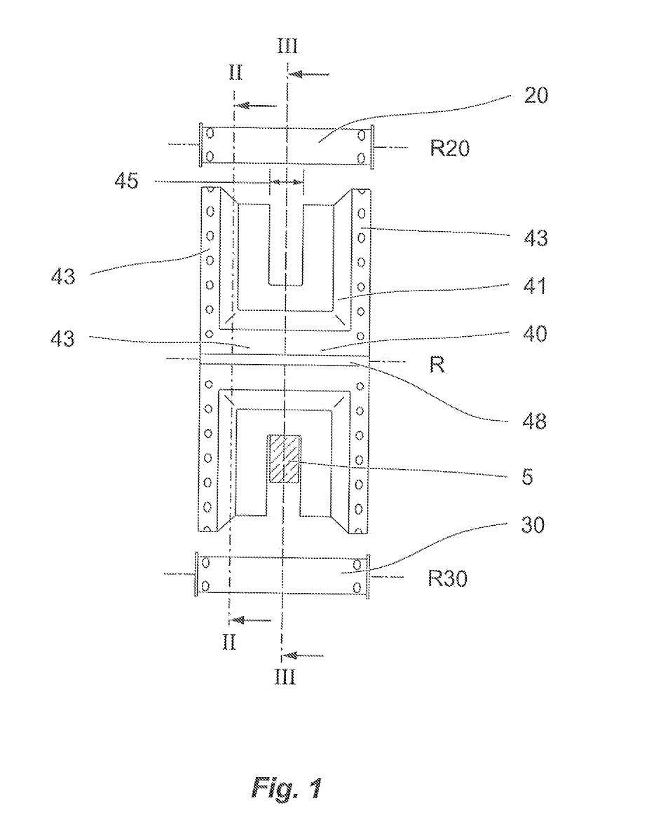

Referring now to the drawings, FIGS. 1-6 show embodiments of a the device. Reference numerals have been assigned to generally refer as follows:
1 Transport device
2 Wrapping
3 Drive element
4 Rear end
5 Ejector
7 Force transmission mechanism
9 Wrapping item
10 Container
12 Fill opening
14 Lid
16 Bearing surface
18 Foot pedal
20 First magazine
22 First wrapping material
23 Spacing
24 First receiver of material stockpile
26 First roll
28 Cup
30 Second magazine
32 Second wrapping material
34 Second receiver of material stockpile
36 Second roll
38 Cup
40 Drum
41 Receiving recess
42 Bottom
43 Sealing zone
44 Inlet radius
45 Central region
46 Outlet radius
47 Driven element
48 Connecting and/or separating device
49 Knob
R Axis of rotation (Drum)
R20 Wrapping rotation axis
R30 Wrapping rotation axis
RR Rotation direction
Z Gusset

FIGS. 1 to 6 show embodiments of a device for enclosing of portions of excrement, bandaging materials, feminine hygiene articles or the like in wrappings. All figures contain the same reference numbers for the same parts. However, to maintain clarity, not all reference numbers are indicated in all figures.

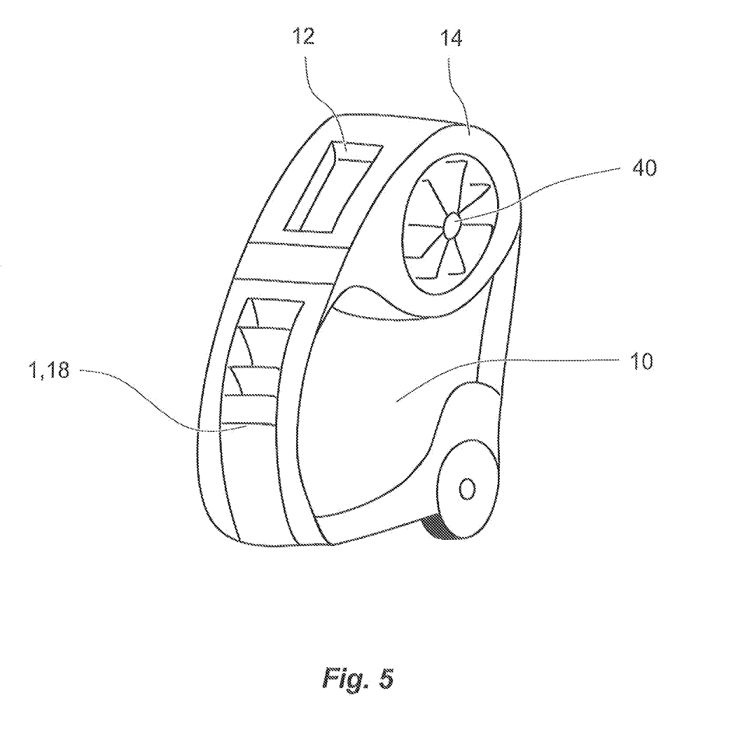

An overview of the device from the outside is shown in FIG. 5. Schematically represented is a container 10 for keeping of wrapped items containing excrement, bandaging materials, feminine hygiene articles, or the like, sealed odor-tight. On top of the container is a lid 14, accommodating functional parts of the device. Beneath this one notices a central body of the device, namely, a drum 40 in simplified and technically imprecise representation and dimensioning.

The perspective view of FIG. 5 moreover allows a glimpse of the front side of the device with the fill opening 12 indicated at the top for filling with wrapping item, and a foot pedal 18 indicated at the bottom for the driving of the drum 40.

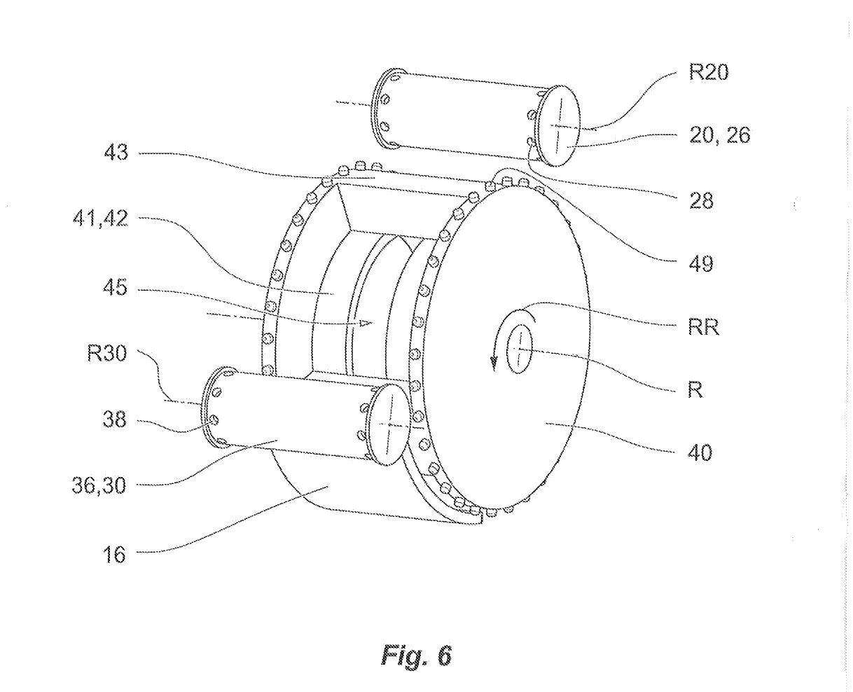

FIGS. 1 and 6 provide a view of the interior of the lid 14. Still only schematically depicted are the drum 40, which can turn by means of the foot pedal 18 about an axis of rotation R. Moreover, one notices a first 20 and a second magazine 30 around its circumference above and below the fill opening 12 depicted in FIG. 5 and coaxial R20, R30 to the axis of rotation R, with radially separated rolls 26, 36 in an exploded view. These are designed to roll along the drum 40. In some embodiments, it may be desirable, for the material transport and for sealing purposes, to structurize the contact surfaces, as indicated by knobs 49 and cups 28, 38.

In the circumferential direction RR of the drum 40 further along the rotation direction RR dictated by the drive unit of the drum 40 is shown in FIG. 6 a bearing surface 16 following the second roll 36 of the second magazine 30 and increasingly applied against the drum 40 in the circumferential direction. This produces a sealing of the wrapping item, not yet shown here. The magazines 20, 30, the drum 40 and the bearing surface 16 are accommodated at least temporarily in the lid 14.

In the scope of the invention, an embodiment is also included which has a reception directly in the container, doing away with a lid. Likewise, the scope of invention includes applications in existing facilities that accommodate the device, for example, in hospital facilities or aircraft furnishings.

Looking more closely through the fill opening 12 of FIG. 5, as shown in FIG. 6, one recognizes a receiving recess 41 for the wrapping item, not yet shown here, with a bottom 42. The bottom 42 is interrupted axially R at the middle in a central region 45, in order to allow a radial passageway for an ejector 5 indicated by hatching in FIG. 1. The passageway lies in the circumferential direction, following the downward direction of rotation RR of the bearing surface 16 (not shown in FIG. 6).

FIG. 1 shows two of the receiving recesses 41 distributed about the drum in the circumferential direction. The receiving recesses in the context of the invention can be of different sizes, in order to allow for different diaper sizes, for example. Between the receiving compartments 41 the circumferential surface is once more at the radial normal dimension, in order to form sealing zones 43. On the whole, the sealing zone 43 runs entirely around the receiving recess 41.

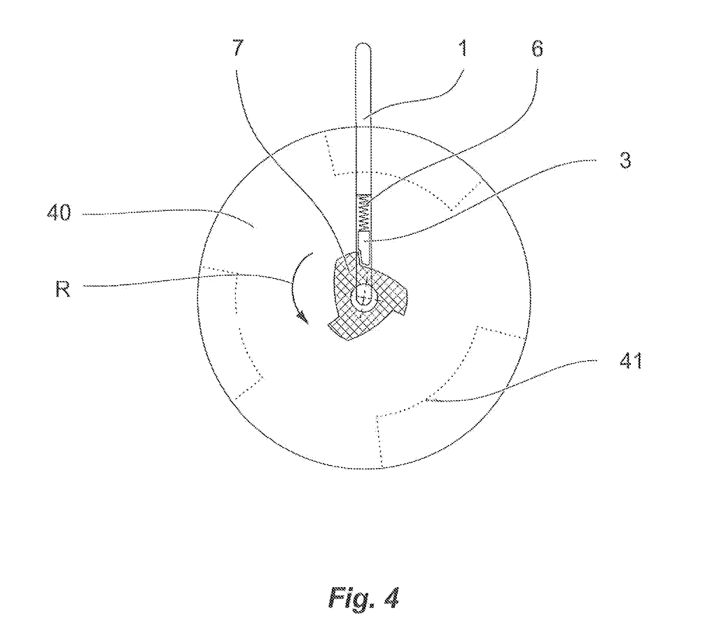

FIG. 1 shows a connecting and/or separating device 48 radially above the axis of rotation R as a groove running axially in the drum rolling surface. A squeegee and/or blade, not shown, is designed to enter this groove radially from the outside as soon as a complete stroke of a transport device 1 (FIG. 4, 5) has occurred, which has further rotated the drum 40 around a receiving recess 41.

The wrapping materials according to the invention can be joined together by folding techniques, and then the blade action even without an actual cutting results in the ejection of a single wrapping. Alternatively according to the invention, in the case of a continuous strip of wrapping material, the blade can produce a perforation, preferably with surface pressure at the same time to improve the sealing action.

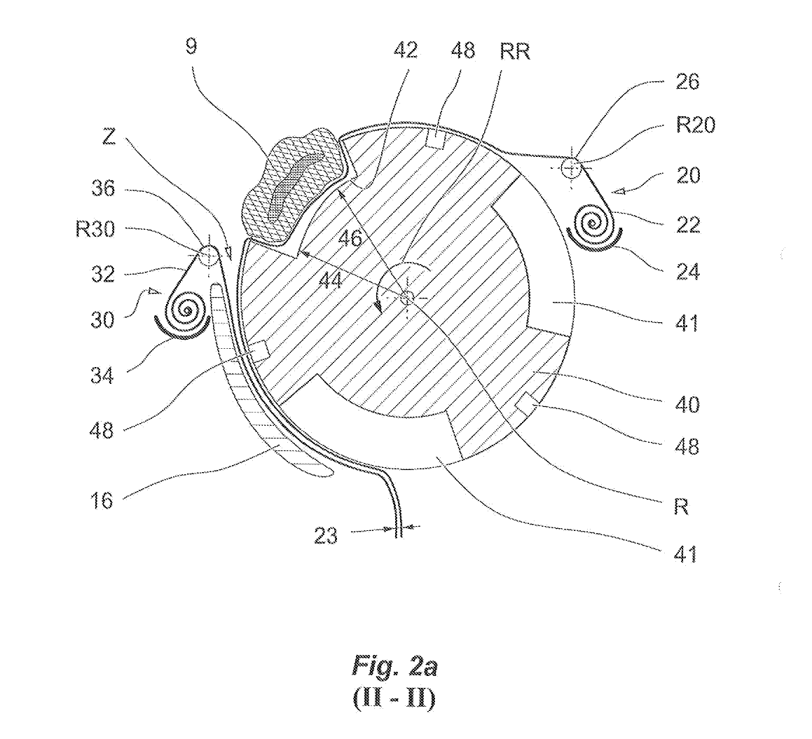

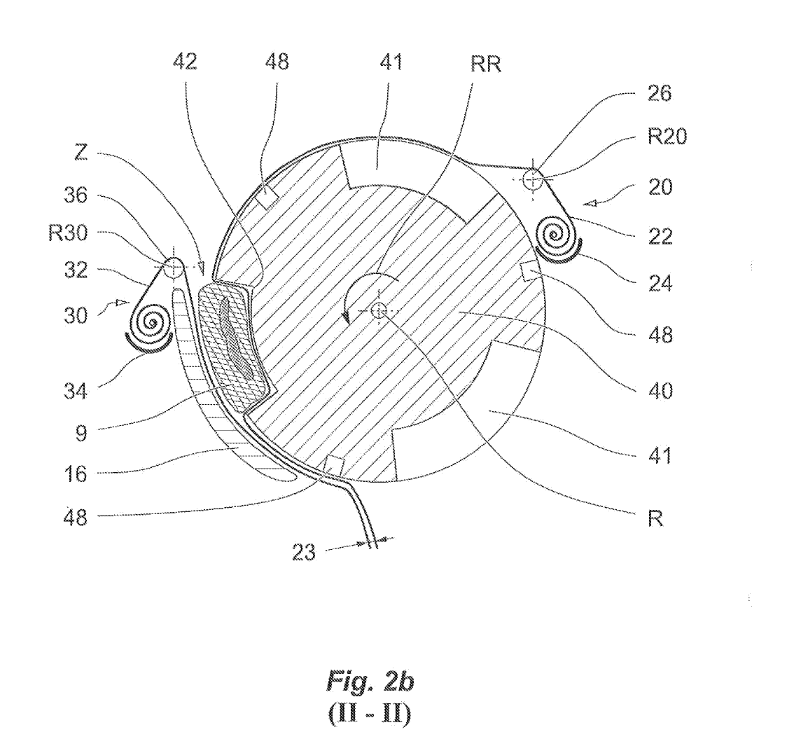

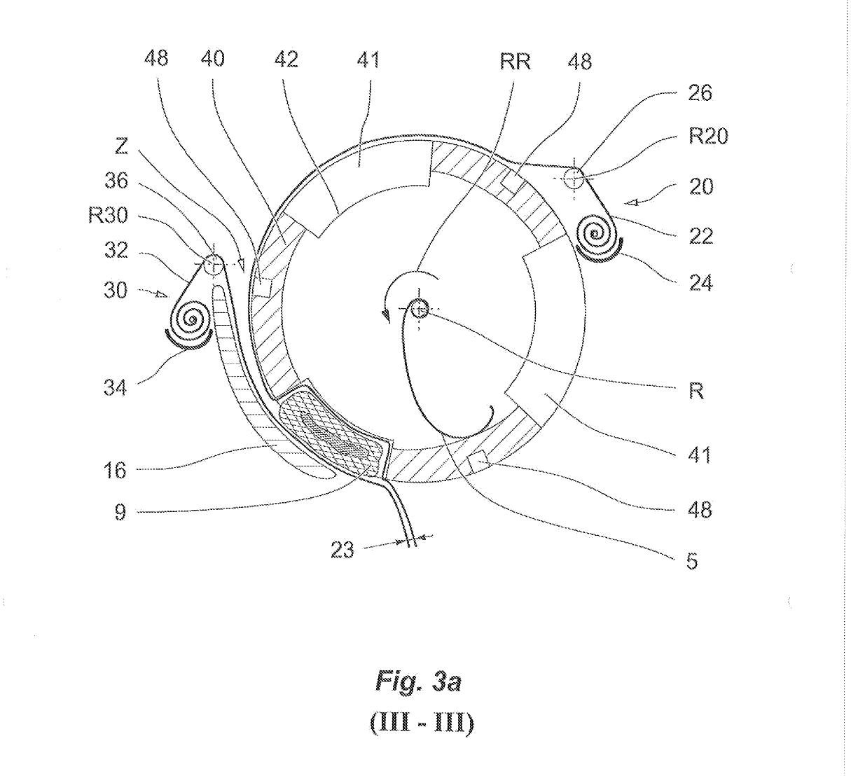

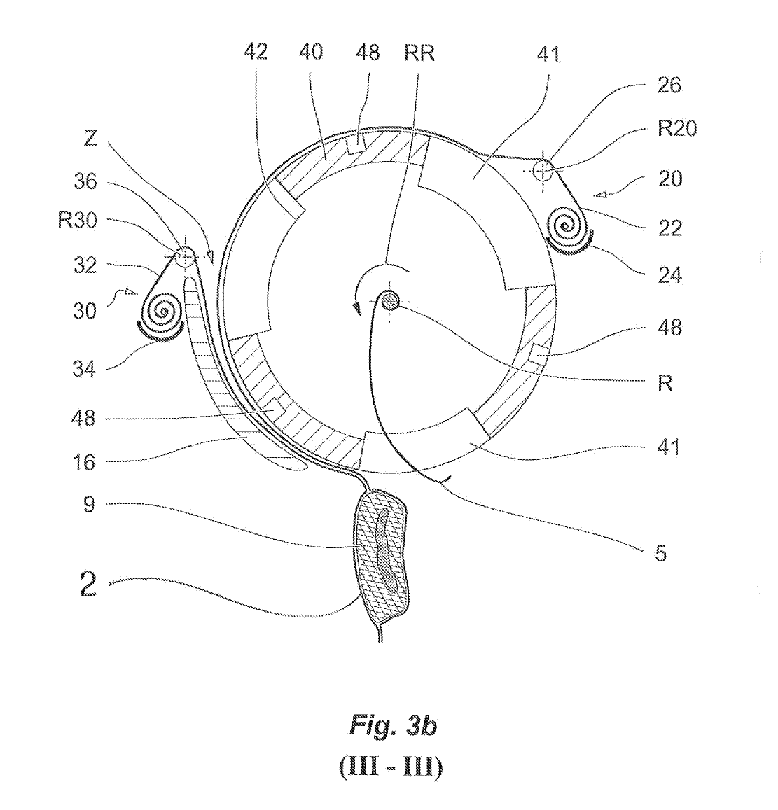

FIGS. 2 and 3, including the respective representations a and b, show a movement of a wrapping item 9 through the device in consecutive steps according to the numbering 2a, 2b, 3a, 3b from the filling 2a to the ejecting 3b.

These representations show in somewhat more detail yet still schematically additional functional parts of the magazines 20, 30. At each magazine 20, 30, wrapping material 22, 32, such as a roll of material, is held in a receiver 24, 34. Receivers, according to embodiments, hold the material stockpile, for example, only on stub axles of an axially protruding winding axle. A material transport of the unwound wrapping material 22, 32 in the direction of the drum 40 is again indicated by rolls 26, 36. However, the representation is not technically correct, because this would not allow an engaging of knobs and cups. In an embodiment of the invention, not shown here, no such rolls 26, 28 are used and the drum 40 alone takes over the transport function for the wrapping material 22, 32.

The wrapping material 22, 32 itself is only indicated by a heavy line. According to embodiments, it can be multi-ply. Moreover, the first wrapping material 22 and the second wrapping material 32 after the described contact in the sealing zone 43 naturally no longer have the indicated spacing 23 from each other. The spacing 23 indicated, but no longer present after the contact, serves merely to illustrate the flow of material.

In the drawings of FIGS. 2 and 3, moreover, one notices that the bottom 42 has a small inlet radius 44 and a large outlet radius 46, in order to assist the sealing process, which occurs per FIG. 2b between bottom 42 and bearing surface 16. During the closure of the wrapping materials 22, 32 around the wrapping item 9, the wrapping item 9 enters a gusset Z. In the position of FIG. 3a, the sealing process is finished and the ejector 5 is tensioned. In FIG. 3b, following its relaxation, it travels through the bottom 42 in the central region in order to assist the ejecting. Here as well, the contact required for this is not shown. The ejector 5 is already fully relaxed in FIG. 3b and the wrapping has long since left the receiving recess 41 downward into the container 10. Upon falling downward, the wrapping is preferably separated by tearing at a perforation.

The foot pedal 18 and a stroke of the transport device 1 have already been described above. A proposed embodiment of such a single-stroke mechanism is shown in FIG. 4. The transport device 1 comprises a lever. The lever can be part of a linkage which interacts with the foot pedal 18. Alternatively, the invention comprises a manual lever (not shown).

The transport device 1 comprises a spring 6, which is designed to act on a drive element 3 movably accommodated in the transport device 1. The drive element 3 is in indirect contact with the drum 40 across a force transmission mechanism 7. The drive element 3 is ratcheted to the force transmission mechanism 7 during each stroke, according to the mechanics shown as an example.

What is claimed is:

1. A device for the enclosing of portions of excrement, bandaging materials, feminine hygiene articles or the like, each in a wrapping, comprising:
a container or a lid for a container with at least one fill opening to receive at least one object being sealed,
a movement coordinator at least partly mounted on the container or the lid for a propelling of the object in at least one direction of movement, at least one magazine for dispensing of a wrapping material, wherein at least one surface region of the movement coordinator is arranged in the direction of movement toward the magazine,
wherein either the fill opening disposed in the direction of movement is arranged between at least two magazines each with at least one wrapping material or the at least one magazine stockpiles at least two wrapping materials, which can be separated from each other before reaching the fill opening or in the fill opening, disposed in the direction of movement,
and a joining device, configured to communicate with the fill opening or which is arranged after the fill opening in the direction of movement, in order to join together the wrapping materials enclosing the object.

2. The device of claim 1,
wherein the movement actuator is a drum, whose axis of rotation is oriented at least approximately horizontally or vertically.

3. The device of claim 1,
wherein the magazines are arranged with parallel wrapping material axis of rotation, wherein the wrapping material axis of rotation is parallel to the axis of rotation of the rotatable drum.

4. The device of claim 1,
wherein at least one gusset is formed between a bearing surface secured or formed in the container or the lid and a segment of a margin enclosing the fill opening.

5. The device of claim 1,
wherein the fill opening is coordinated with at least one bottom arranged in the movement coordinator, which can be locked by being arranged roughly congruent to the surface of the fill opening, depending on the position of the movement coordinator.

6. The device of claim 5,
wherein the bottom when viewing a cross section along the direction of movement is arranged to narrow the fill opening in the direction of movement, especially in the case when the movement coordinator is configured as a drum with a smaller inlet radius and a larger outlet radius.

7. The device of claim 1, wherein the wrapping material is completely gas-tight or at least outfitted to be gas-tight.

8. The device of claim 1,
wherein the movement coordinator itself has receiving recesses bordered by a plurality of sealing zones distributed about its surface, while according to a preferred embodiment the sealing zones are partial surfaces of an imaginary circumferential surface of the drum.

9. The device according to claim 4,
wherein a pressure roll of the second magazine is a bearing surface for the sealing of the object and a sealing pressure surface at the same time.

10. The device of claim 1,
wherein in a central region of the movement coordinator at least one ejector is arranged and the movement coordinator is at least partly open in a middle travel region, so that the ejector coming after the second magazine in the direction of movement reaches the wrapped material and ejects it, and preferably the wrapped material is designed to deflect the ejector against the force of a spring.

11. The device of claim 1, wherein the container has at least one of the following features:
can be sealed gas-tight,
can be closed, in particular, with a combination lock,
containing a counter and an interface for data transmission,
having a display to show the full amount and/or danger of odor,
having a camera,
having a trap bowl for moisture, especially with an odor blocking layer, especially glycerin,
double-walled with absorption material between the walls,
having a lighting fixture and an energy storage, the energy storage being chargeable during the driving of the drum and/or by solar energy,
having an atmosphere on the inside, especially a sterilizing gas,
having a trap door with clamping lip as a seal,
having a window with magnifying glass for intensive illumination of the container volume.

12. A wrapping method for the enclosing of portions of objects such as excrement, bandaging materials, feminine hygiene products or the like, in wrappings, comprising:
a transport device for a first gas-tight wrapping material, which together with the first wrapping material is designed to form a receiving recess,
a magazine having a second gas-tight wrapping material, wherein preferably a single magazine stockpiles both wrapping materials,
a sealing mechanism, which is designed to seal the object gas-tight by at least bringing both wrapping materials into contact, and
a movable body for the closing process, wherein
a single movement of the body first of all removes the first wrapping material from a first magazine, secondly seals the object placed in the receiving recess and thirdly removes the second wrapping material from the [first magazine] or a second magazine for the sealing and brings both wrapping materials into contact in a sealing zone around the receiving recess.

13. The wrapping method of claim 12,
wherein a cold welding takes place completely around the receiving recess in the sealing zone in order to block off the sealed wrapping item hermetically from the surroundings.

14. The wrapping method of claim 12,
wherein the sealing coming after the wrapping process is at first intensified by a shrinkage process, wherein said shrinkage is triggered by a contact between wrapping material and wrapping item, and a gas tightness of the wrapping materials occurs only after the shrinkage process.

15. The wrapping method of claim 12, wherein a single driving stroke is required for each portion of wrapping item being closed, acting upon the transport device.

16. The wrapping method of claim 12, wherein the driving of the body entrains an active evacuating mechanism, especially a pump, and a connection between the wrapping and the evacuating mechanism remains open until a rear end of the wrapping is sealed.

17. The device of claim 1, wherein all materials used for the wrapping are biodegradable.

18. The wrapping method of claim 12, wherein all materials used for the wrapping are biodegradable.

19. The device of claim 1, wherein the object comprises an energy-rich material.

20. The method of claim 12, wherein the object comprises an energy-rich material.
